(19) United States
(12) Patent Application Publication
(10) Pub. No.: US 2023/0120360 A1
(43) Pub. Date: Apr. 20, 2023

(54) TRADITIONAL CHINESE MEDICINE-BASED AGENT FOR CACHEXIA PREVENTION AND TREATMENT

(71) Applicant: CHINA MEDICAL UNIVERSITY, Taichung City (TW)

(72) Inventors: Hsiang-Wen Lin, Taichung City (TW); Chih-Hsueh Lin, Taichung City (TW); Liang-Yo Yang, Taipei City (TW); Chih-Shiang Chang, Taichung City (TW); Ching-Shih Chen, Taipei City (TW)

(73) Assignee: CHINA MEDICAL UNIVERSITY, Taichung City (TW)

(21) Appl. No.: 18/046,503

(22) Filed: Oct. 13, 2022

Related U.S. Application Data
(60) Provisional application No. 63/255,963, filed on Oct. 15, 2021.

(51) Int. Cl. A61K 36/65; A61P 21/00; A61P 35/00

(52) U.S. Cl. CPC A61K 36/65; A61P 21/00; A61P 35/00; A61K 2236/333

(57) ABSTRACT

This invention discloses a traditional Chinese medicine, Mu Dan Pi (Moutan radicis cort), that has the potential to be used for the prevention or treatment of cachexia and muscle loss in cancer patients. The composition is a novel therapeutic agent for cachexia.

### CROSS-REFERENCE TO RELATED APPLICATION

This application claims the benefit of U.S. Provisional Patent Application No. 63/255,963, filed Oct. 15, 2021, which is incorporated by reference herein in its entirety.

This application contains a Sequence Listing in a computer readable form, the file name is 3991-CMU-SEQ1013, created on Oct. 13, 2022, the size is 24 KB, which is incorporated herein by reference in its entirety.

### FIELD OF THE INVENTION

The present invention discloses a traditional Chinese medicine (TCM)-based agent, Mu Dan Pi (MDP) (Moutan radicis cort), which can be used to prevent or treat tumor-induced muscle atrophy and cachexia in cancer patients. This agent is developed through an integrated, multi-tiered strategy involving both in vitro and in vivo muscle atrophy platforms from an in-house TCM library.

### BACKGROUND OF THE INVENTION

A major challenge in the treatment of certain types of cancers, including those of pancreas, stomach, lung and colon, is the accompanying cancer-induce body weight loss, which is characterized by anorexia and loss of adipose tissues and skeletal muscle masses, in terms of cachexia. Given no broad consensus on definitions of cancer cachexia, the present of at least three features among the following five characteristics was considered as cachexia: decreased muscle strength, fatigue, anorexia or limited food intake, low fat-free mass index and abnormal biochemistry (e.g., increasing C-reactive protein, anemia, or low serum albumin), in addition to edema-free weight loss 5% (or a body mass index (BMI)<20.0 kg/m² in 12 months. As a result, the body weight loss due to cancer cachexia cannot be reversed by nutritional support, and has severe impacts on the morbidity, mortality, and quality of life of cancer patients. Although substantial advances have been made in understanding the multifactorial pathophysiology of cachexia, prevention and/or treatment of this debilitating disease remains an unmet medical need.

Currently, no approved targeted therapy is available for cachexia treatment. The semi-synthetic progestational steroid, megestrol, is used to ameliorate cachexia-associated symptoms.

In addition, a number of Kampo medicines (i.e., multi-component herbal extracts) have been commonly prescribed in Japan to alleviate fatigue and chronic weakness in cachexia patients, which act upon the immune system to improve inflammatory and nutritional status. More recently, although the hunger hormone ghrelin and ghrelin mimetics have received much attention in light of their potential to enhance appetite and quality of life, clinical evidence is lacking to support their use for the treatment of cachexia.

The advantage of TCMs over small-molecule targeted agents for the prevention and/or treatment of cachexia is multifold. First, the therapeutic utility of the polypharmacology (or network pharmacology) of TCMs is manifested by their long-standing history in the treatment of various chronic and complex diseases. Second, many TCMs could be consumed as dietary supplements on a daily basis for disease control and prevention. Third, TCMs are generally perceived in oriental societies as having fewer side effects, which might lead to better compliance in cancer patients with muscle atrophy.

### SUMMARY OF THE INVENTION

In view of the above technical circumstances, the present invention provides a TCM composition for the treatment and/or prevention of cachexia with a definite clinical benefit, preparations thereof, and a method for preparing the same.

This invention discloses a TCM, Mu Dan Pi (MDP) that has the potential to be used for the prevention or treatment of cachexia in cancer patients. The MDP extract is prepared by soaking the TCM in 50% to 100% methanol or 50% to 100% ethanol.

The present invention also provides a method of treating or preventing cancer-induced cachexia, comprising the administration of effective amounts of the composition to a subject with tumor-associated cachexia, wherein the composition comprises a Moutan radicis cort or a Moutan radicis cort extract. The extract of MDP is prepared by soaking the TCM in 50% to 100% methanol or 50% to 100% ethanol.

The present invention further provides a composition for preventing or treating skeletal muscle atrophy in cancer patients, which is attributable to its ability to reverse tumor-induced reprogramming of muscle homeostasis-associated gene expression in skeletal muscles, thereby rescuing skeletal muscles from wasting. The extract of MDP is prepared by soaking the TCM in 50% to 100% methanol or 50% to 100% ethanol.

The present invention also provides a method treating or preventing cancer-induced losses of skeletal muscle mass, comprising the administration of effective amounts of the composition to a subject with tumor-associated skeletal muscle atrophy, which is attributable to its ability to reverse tumor-induced reprogramming of muscle homeostasis-associated gene expression in skeletal muscles, thereby rescuing skeletal muscles from wasting, wherein the composition comprises the Moutan radicis cort or extracts of Moutan radicis cort. The extract Mu Dan Pi (MDP) is prepared by soaking the TCM in 50% to 100% methanol or 50% to 100% ethanol.

### BRIEF DESCRIPTION OF THE DRAWINGS

FIGS. 1A-1C illustrate the effects of DMSO and different TCMs on C26CM-induced C2C12 myotube atrophy. FIG. 1A illustrates the experimental procedure for C26CM-induced atrophy of C2C12 myotubes. FIG. 1B illustrates the effects of DMSO and H3-14 on C26CM-induced atrophy of C2C12 myotubes. FIG. 1C illustrates myotube diameter of C26CM-induced C2C12 treated with different TCMs.

FIG. 2 illustrates the lack of protective effect of 24 TCM extracts on C26CM-induced atrophy of C2C12 myotubes.

FIGS. 3A-3D illustrate the time-dependent effects of MDP versus DR on age-associated mobility and/or total body contraction in C. elegans. FIG. 3A illustrates the mobility of C. elegans treated with DR. FIG. 3B illustrates the mobility of C. elegans treated with MDP. FIG. 3C illustrates the mobility of C. elegans treated with 100 μg/ml MDP. FIG. 3D illustrates the relative total body contraction in C. elegans treated with 100 μg/ml MDP.

FIGS. 4A-4E illustrate the effects of MDP with three different doses (MDP-L, MDP-M, and MDP-H) versus vehicle via oral gavage on the body weight, tumor volume, protecting hindlimb muscles of C-26 tumor-bearing mice, and the alert and active phenotype of C-26 tumor-bearing mice. FIG. 4A illustrates the change of body weight of C-26 tumor-bearing mice treated with three different doses of MDP. FIG. 4B illustrates the change of body weight w/o tumor of C-26 tumor-bearing mice treated with three different doses of MDP.

FIG. 4C illustrates the tumor volume of C-26 tumor-bearing mice treated with three different doses of MDP. FIG. 4D illustrates mean of mass normalized to control on three different sections of skeletal muscles (Quad, GC, and TA) of hindlegs of C-26 tumor-bearing mice treated with three different doses of MDP. FIG. 4E illustrates the alert and active phenotype of C-26 tumor-bearing mice treated with three different doses of MDP.

FIGS. 5A-5C illustrate the effects of DR with 100 mg/kg versus vehicle via oral gavage on body weight (w/o tumor) and tumor volume. FIG. 5A illustrates the change in body weight w/o tumor of C-26 tumor-bearing mice treated with 100 mg/kg DR. FIG. 5B illustrates the photograph of C-26 tumor-bearing mice treated with three different treatments (w/o tumor, vehicle, and DR). FIG. 5C illustrates tumor volume of C-26 tumor-bearing mice treated with 100 mg/kg DR.

FIGS. 6A-6F illustrate a duplicate experiment showing the effects of MDP with 1000 mg/kg (MDP-H) versus vehicle via oral gavage on the body weight, tumor volume, its ability to diminish cachexia-associated decreases in skeletal muscle weights, rescuing the fiber size distribution from shifting to smaller cross-sectional areas, and restore the forelimb grip strength at day 17 of C-26 tumor-bearing mice. FIG. 6A illustrates the change in body weight of C-26 tumor-bearing mice treated with 1000 mg/kg MDP (MDP-H). FIG. 6B illustrates the change in body weight w/o tumor of C-26 tumor-bearing mice treated with 1000 mg/kg MDP (MDP-H). FIG. 6C illustrates tumor volume of C-26 tumor-bearing mice treated with 1000 mg/kg MDP (MDP-H). FIG. 6D illustrates mean of mass normalized to control on three different sections of skeletal muscles (Quad, GC, and TA) of hindlegs of C-26 tumor-bearing mice treated with 1000 mg/kg MDP (MDP-H). FIG. 6E illustrates muscle fiber size of C-26 tumor-bearing mice treated with 1000 mg/kg MDP (MDP-H). FIG. 6F illustrates the forelimb grip strength of C-26 tumor-bearing mice treated with 1000 mg/kg MDP (MDP-H).

FIGS. 7A-7C illustrate the effects of MDP on serum IL-6 in Veh-versus MDP-H-treated C26-tumor-bearing mice and its cell viability to C26 cells. FIG. 7A illustrates the mean serum IL-6 levels in the serum of C26 tumor-bearing mice treated with 1000 mg/kg MDP (MDP-H). FIG. 7B illustrates the secretion of serum IL-6 level by C26 cells treated with three treatments (DMSO, 25 μg/ml MDP, and 50 μg/ml MDP). FIG. 7C illustrates cell viability of C26 cells treated with three treatments (DMSO, 25 μg/ml MDP, and 50 μg/ml MDP).

FIGS. 8A-8B illustrate the bioinformatic analysis of shotgun sequencing. FIG. 8A illustrates two dimensional projection of RNA-seq data from the three study groups (T/Veh, TF/Veh, T/MDP). FIG. 8B illustrates a Venn diagram of total differentially expressed genes from the three study groups (T/Veh, TF/Veh, T/MDP).

FIGS. 9A-9B illustrate the effects of MDP on skeletal muscle-related genes and protein expressions. FIG. 8A illustrates the relative expression level of different skeletal muscle-related genes and proteins from MDP-H-treated mice. FIG. 9B illustrates western blot analysis of the protein expression levels of MuRF1 and Atrogin-1 in skeletal muscles from vehicle- and MDP-H-treated mice (T/Veh, TF/Veh, T/MDP).

### DETAILED DESCRIPTION OF THE INVENTION

As used herein, “a,” “an,” “the,” “at least one,” and “one or more” are used interchangeably.

In one embodiment, the DR and MDP showed their abilities to suppress the inflammatory cytokine-induced atrophy effect of C2C12 myotubes.

In one embodiment, MDP ameliorates age-associated decreases in the mobility of C. elegans.

In one embodiment, the MDP prevents tumor-induced muscle wasting in C26 tumor-bearing mice.

In one embodiment, the MDP is effective in protecting hindlimb muscles, including quadriceps and tibialis anterior, against cancer-induced wasting.

In one embodiment, the MDP shows in vivo efficacy in protecting mice from C-26 tumor-induced body weight loss.

In another embodiment, the MDP diminishes cachexia-associated decreases in skeletal muscle weights.

In one embodiment, the MDP is able to rescue the fiber size distribution from shifting to smaller cross-sectional areas in cachectic muscles (P<0.05).

In one embodiment, the MDP reduces serum IL-6 levels.

In one embodiment, the MDP exerts the anti-cachectic effect by reversing tumor-induced reprogramming of muscle homeostasis-associated gene expression in skeletal muscle.

The present invention provides a method of treating or preventing cancer-induced cachexia, comprising the administration of effective amounts of a composition to a subject with cancer-induced cachexia, wherein the composition comprises a Moutan radicis cort or a Moutan radicis cort extract.

The present invention also provides a method of treating or preventing cancer-induced skeletal muscle weight losses, comprising the administration of effective amounts of a composition to a subject with cancer-induced muscle atrophy, wherein the composition comprises a Moutan radicis cort or a Moutan radicis cort extract.

In one embodiment, the Moutan radicis cort extract is prepared by soaking the Moutan radicis cort in 50% to 100% methanol or 50% to 100% ethanol.

In a preferred embodiment, the Moutan radicis cort extract is prepared by soaking the Moutan radicis cort in 70% methanol or 70% ethanol.

### Example

The examples below are non-limited and are merely representative of various aspects and features of the present invention.

### Example 1: C26CM-Induced Myotube Atrophy Model

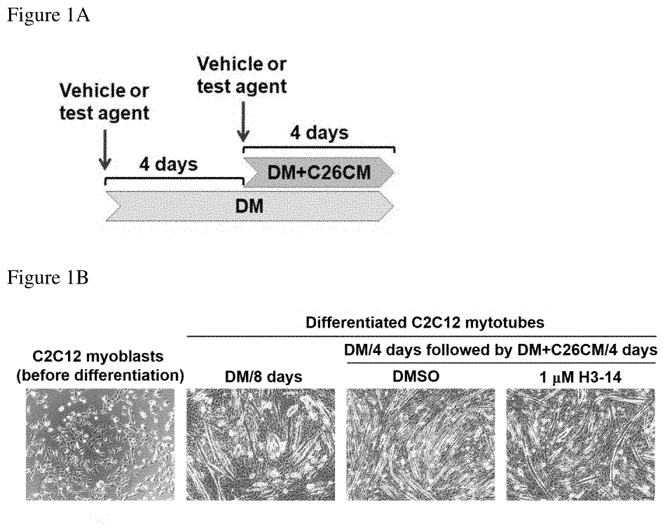

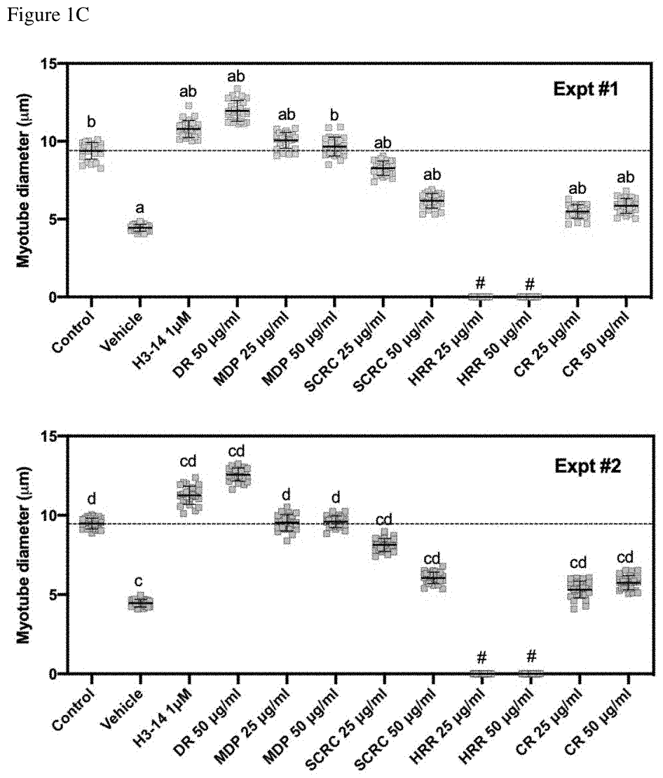

The experimental design is depicted in FIG. 1A C2C12 myoblasts are exposed to 2% horse serum-containing differentiation medium (DM) for 4 days to facilitate their differentiation into myotubes, followed by treatment with C26CM for 4 days. At the end of this 8-day treatment, diameters of C26CM-treated C2C12 myotubes versus those of control cells (receiving DM only) are analyzed under light microscopy, of which the difference was quantified as a readout of C26CM-induced atrophy. In order to recapitulate the use of TCMs to delay the onset of muscle wasting, individual TCMs versus DMSO are added to culture media from the beginning throughout the course of experiment with daily replacement (H3-14 as another control), each of which was repeated four times. As shown, C26CM caused significant narrowing in C2C12 myotubes (FIG. 1B & FIG. 1C).

As shown in FIG. 1, the effects of different TCMs on C26CM-induced C2C12 myotube atrophy was shown. Schematic representation of the experimental design was shown in FIG. 1A. FIG. 1B was the image. FIG. 1C were the two quantitative analyses of image of FIG. 1B about the protective effect of Dioscoreae rhizome (DR), MDP, and three other representative TCMs on C26CM-induced atrophy of C2C12 myotubes of the representative experiments. Bar, means±S.D. (for data from two independent experiments Expt #1 and Expt #2).

Extracts of different TCMs, each at 25 and/or 50 μg/ml, were evaluated for their anti-atrophy activities, including Dioscoreae rhizome (DR), MDP, Sambuci chinensis radix et caulis (SCRC), Helminthostachydis radix et rhizome (HRR), Condonopsis radix (CR), Polygonati odorati rhizome, Glycyrrhizae radix et rhizome, Lilii bulbus, Citri sarcodactylis fructus, Euryales semen, Hordei fructus germinates, Siraitiae fructus, Pruni semen, Lycii fructus, Poria, Platycodonis radix, Bombycis chrysallidem, Alpiniae oxyphyllae Fructus, Nelumbinis semen, Polygonati rhizome, Sesami nigrum semen, Ziziphi spinosae semen, Coicis semen, Rubi fructus, Ginseng radix et rhizome, Amynthas et metaphire, Arctii radix, Portulacae herba, and Trionycis carapax. Among these TCM extracts, we found two widely used TCMs, DR and MDP, shared the ability of H3-14 to fully protect C2C12 myotubes from C26CM-induced atrophy (all Ps=0.0002 vis-à-vis C26CM control in the myotube atrophy platform, FIG. 1C), but others were either cytotoxic, or provided no protection, of which the representative data were shown in FIG. 1C.

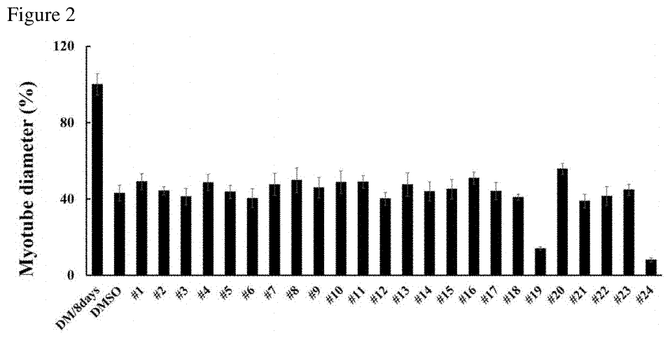

Data of other TCMs were shown in FIG. 2. Bar, means±S.D. (N=4). #1 was Polygonati odorati rhizoma; #2 was Glycyrrhizae radix et rhizome; #3 was Lilii bulbus; #4 was Citri sarcodactylis fructus; #5 was Euryales semen; #6 was Hordei fructus germinates; #7 was Siraitiae fructus; #8 was Pruni semen; #9 was Lycii fructus; #10 was Poria; #11 was Platycodonis radix; #12 was Bombycis chrysallidem; #13 was Alpiniae oxyphyllae fructus; #14 was Nelumbinis semen; #15 was Polygonati rhizoma; #16 was Sesami nigrum semen; #17 was Ziziphi spinosae semen; #18 was Coicis semen; #19 was Rubi fructus; #20 was Ginseng radix et rhizoma; #21 was Amynthas et metaphire; #22 was Arctii radix; #23 was Portulacae herba; #24 was Trionycis carapax.

### Example 2: C. elegans Mobility Testing of DR and MDP

In this phenotypic assay of nematode C, elegans, MDP and DR were dissolved in 1% DMSO-containing water at 10 mg/ml as stock solutions, and 100 μl of individual solutions versus vehicle control were evenly applied onto nematode growth medium (NGM) agar plates containing OP50 lawns (total volume of agar, 10 ml). After the solution was completely absorbed into agar, these OP50 plates were radiated under UV for 40 min, followed by seeding with about 50 synchronized eggs of CF512 worms. These plates were incubated at 25° C., and worm mobility was determined starting day 1 after adulthood every other day till day 7. Data, means±SEM. (n=170-420). *P<0.05; **P<0.01; ***P<0.0001 (t-test). The worms at different ages (day 1, 5, 9, 13 adults) were first incubated in drug-free solution and then in levamisole-containing solution for 10 minutes. The digital imaging system were used to quantify the length of the worm body using ImageJ. Data, means±SEM. (n>50). *P<0.05; **P<0.01; ***P<0.0001 (t-test).

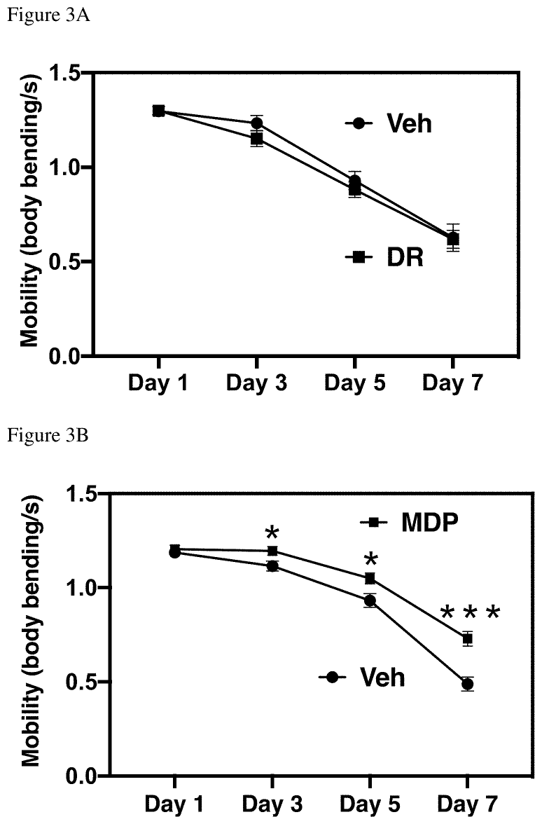

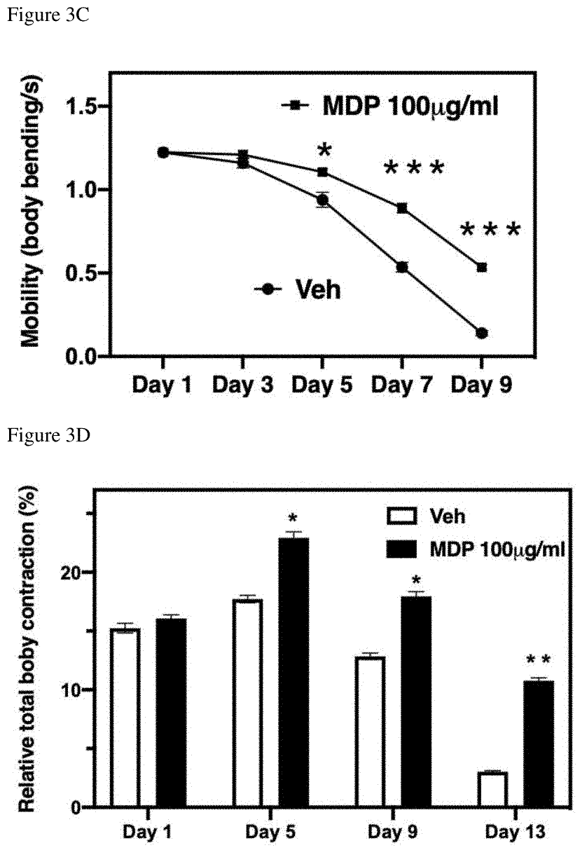

As shown in FIG. 3A, there is no difference of time-dependent effects of Dioscorea radix (DR) as compared to control (vehicle) on age-associated mobility and/or total body contraction in C. elegans.

As shown in FIG. 3B, MDP could ameliorate age-associated decreases in C. elegans mobility relative to control.

As shown in FIG. 3C, the time-dependent effects of MDP showed parallel protective effects of mobility in a separate experiment with longer life-expectancy (body bending/second on day 1, 3, 5, 7 and 9).

As shown in FIG. 3D, MDP significantly delayed the age-associated loss of total body contraction (%) in muscle functions (relative total body contraction on day 1, 5, 9 and 13).

To sum up, MDP exhibited a unique ability to ameliorate age-associated decreases in worm mobility relative to control, but not for DR.

### Example 3: MDP Efficacy in the C26 Model of Cancer Cachexia

The C-26 model is to confirm the in vivo anti-muscle wasting efficacy of MDP versus DR for their abilities to protect CD2F1 mice from C-26 tumor-induced body weight loss, which was reported to be associated with excessive IL-6 secretion by the tumor.

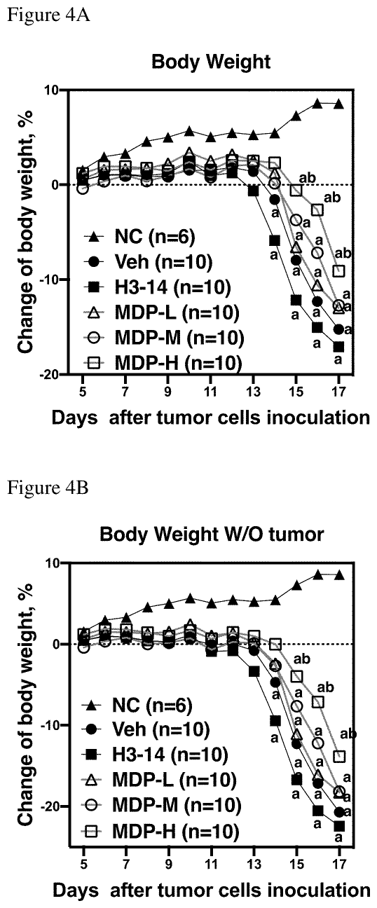

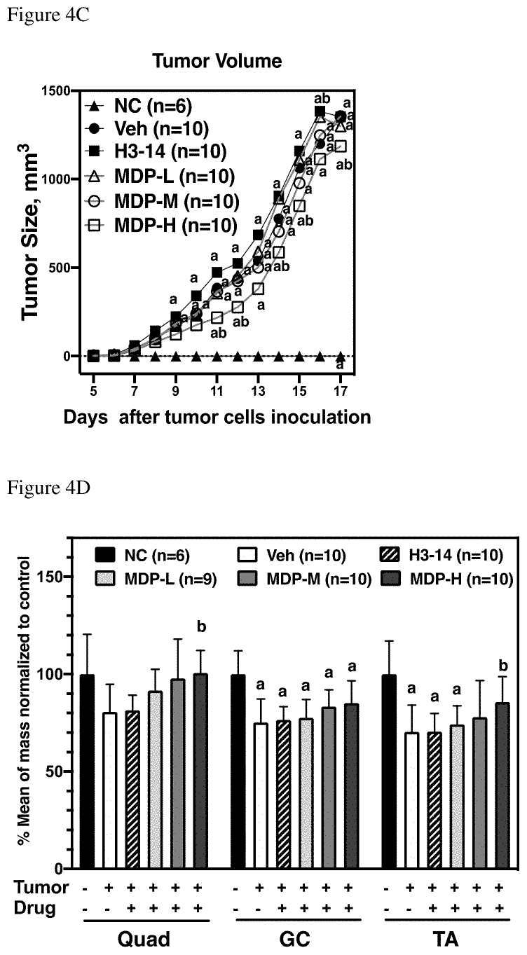

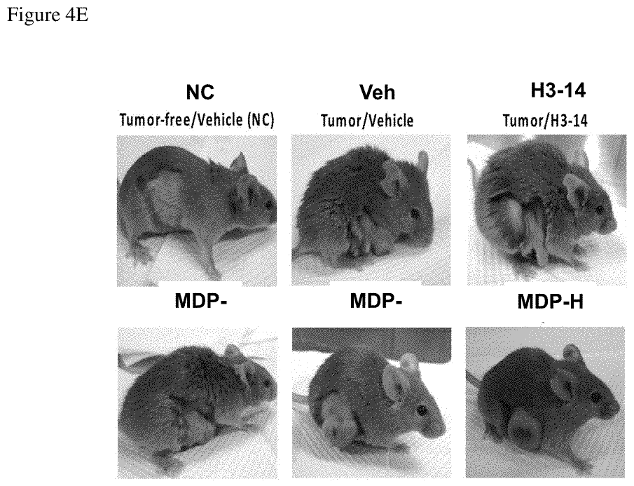

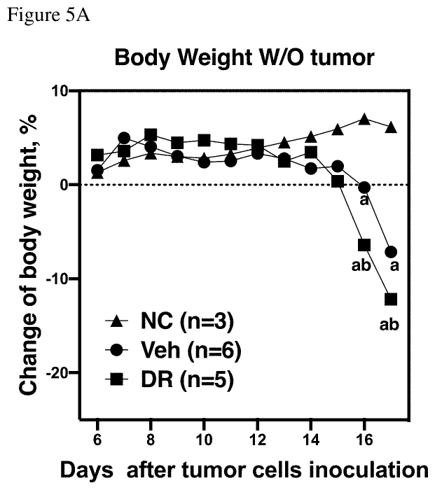

In the first set of experiments, the in vivo efficacy of MDP at three different doses (low dose: MDP-L, 100 mg/kg; medium dose: MDP-M, 500 mg/kg; high dose: MDP-H, 1,000 mg/kg) was evaluated via oral gavage once daily to CD2F1 mice starting at 7 days before C-26 tumor cells were implanted till mice were sacrificed at day 17. The body weight, tumor size, and food and water consumption of individual mice were measured every other day. At sacrifice, hindleg skeletal muscles were dissected and stored at −80° C. for further analysis after the weights were measured. The first set of experiment was shown in FIG. 4.

FIG. 4 illustrates the effects of MDP at three doses (MDP-L, MDP-H, and MDP-H), vehicle and control (H3-14) via oral gavage on the body weight (FIG. 4A & FIG. 4B) and tumor volume (FIG. 4C) of C-26 tumor-bearing mice. NC, tumor-free mice. Tumor weights were estimated based on the assumption that 1,000 mm³ equals to 1 grain of body weight. S.D. bars are not shown to avoid over congestion of these graphs. For statistical analysis, generalized linear mixed-effects models with random intercept for individual subject and fixed effects for treatment and days of the treatment were used to test for differences among groups, using the Tukey-Kramer correction for multiple comparisons. (a) Significant difference from the control group at p<0.05. (b) Significant difference from the vehicle group at p<0.05. In FIG. 4D, the effects of MDP at three doses, vehicle and H3-14 via oral gavage on three different sections of skeletal muscles of hindlegs of C-26 tumor-bearing mice was shown. NC, tumor-free mice. (a) and (b) denote significant difference from NC group and vehicle group, respectively (Kruskal-Wallis test, with Dunn's multiple-comparison test using Bonferroni adjustment, p<0.05). FIG. 4E shown the photographs of one representative mouse from these five groups at the study endpoint depicting the therapeutic effect of MDP-H and MDP-M in tumor-bearing mice, as shown by alertness, normal posture, smooth haircoat, and better body conditions, despite large tumor burden.

As shown in FIG. 4A, MDP-H was effective in ameliorating body weight losses in C-26 tumor-bearing mice.

As shown in FIG. 4B, MDP-H was effective in ameliorating body weight losses (without tumor) in C-26 tumor-bearing mice.

As shown in FIG. 4C, MDP-H showed a very modest tumor-suppressive effect on tumor growth.

As shown in FIG. 4D, MDP-M was effective in protecting hindlimb muscles C-26 tumor-bearing mice.

As shown in FIG. 4E, C-26 tumor-bearing mice treated with MDP-M and MDP-H exhibited an alert and active phenotype.

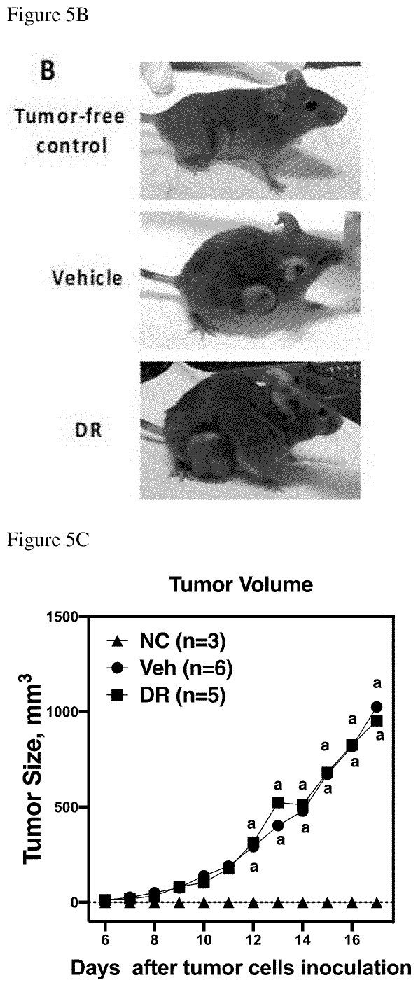

In the second set of experiments, the in vivo efficacy of DR at 100 mg/kg versus vehicle via oral gavage was evaluated, which was shown in FIG. 5.

FIG. 5 illustrated the effects of DR at 100 mg/kg versus vehicle via oral gavage on body weight (w/o tumor) (FIG. 5A) and tumor volume (FIG. 5C) of C-26 tumor-bearing mice (*P<0.05; n=3-6). Control, tumor-free mice. (FIG. 5B) Photographs of representative mice from each group at the study endpoint depicting lack of therapeutic effect of DR. (a) and (b) denote significant difference from the control group and the vehicle group, respectively (Generalized linear mixed-effects models with Tukey-Kramer correction for multiple comparisons).

As shown in FIG. 5A, DR at 100 mg/kg exacerbated body weight loss.

As shown in FIG. 5B, DR at 100 mg/kg caused deterioration of physical appearances relative to vehicle control.

As shown in FIG. 5C, DR at 100 mg/kg had no effect on tumor growth.

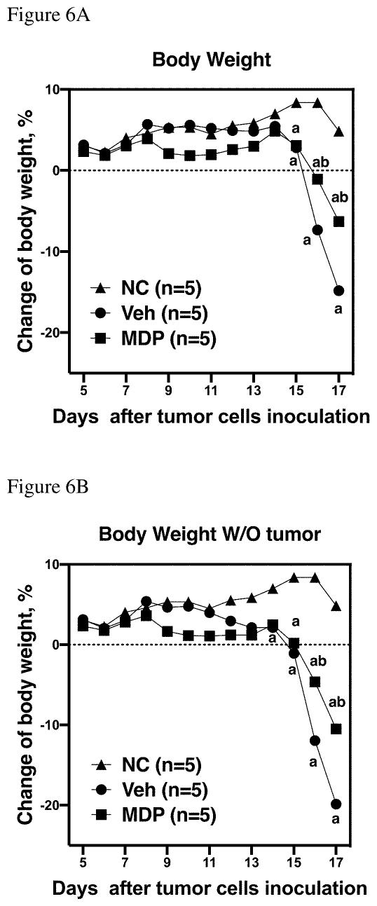

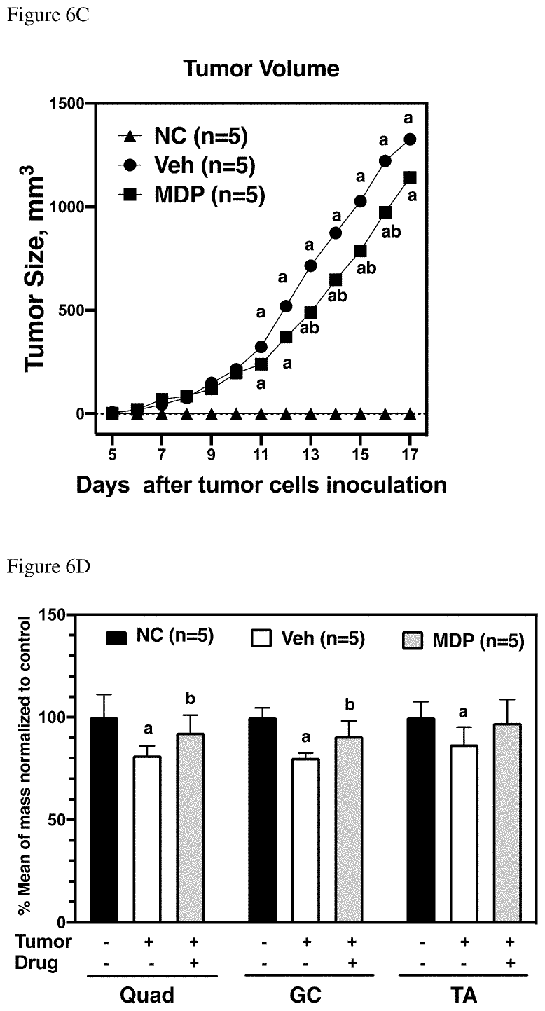

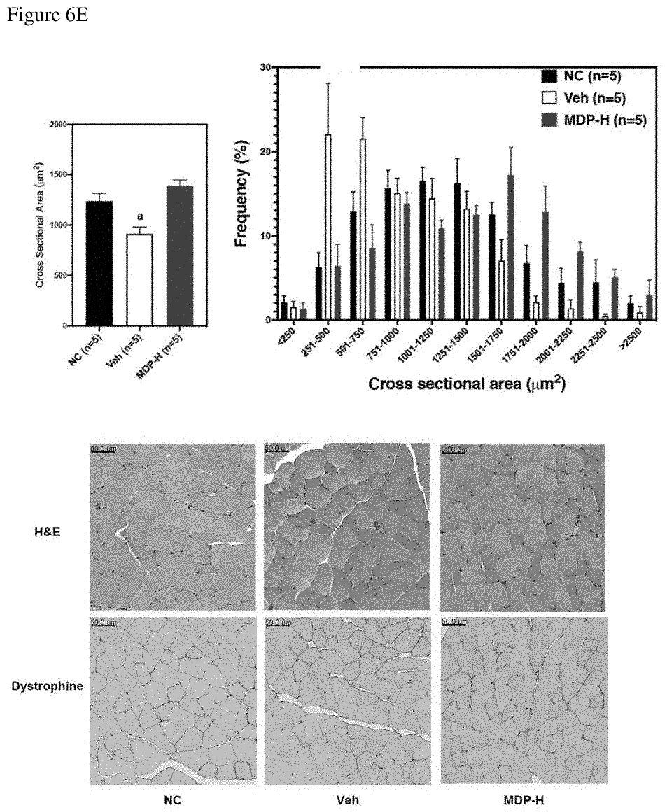

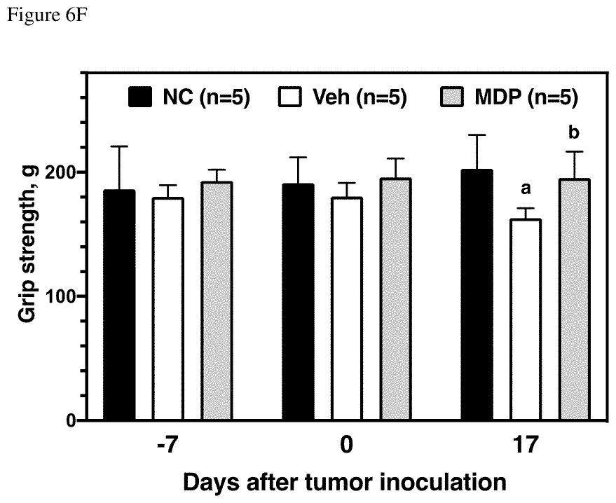

FIG. 6 illustrated a duplicate experiment showing the effects of MDP at 1000 mg/kg (MDP-H) versus vehicle via oral gavage on the body weight (FIG. 6A & FIG. 6B), tumor volume (FIG. 6C) of C-26 tumor-bearing mice. NC, tumor-free mice. Tumor weights were estimated based on the assumption that 1,000 mm³ equals to 1 grain of body weight. S.D. bars are not shown to avoid over congestion of these graphs. For statistical analysis, generalized linear mixed-effects models with random intercept for individual subject and fixed effects for treatment and days of the treatment were used to test for differences among groups, using the Tukey-Kramer correction for multiple comparisons. Significant difference from the control group at p<0.05. (b) Significant difference from the vehicle group at p<0.05. FIG. 6D shown the effects of MDP-H versus vehicle on three different sections of skeletal muscles of hindlegs of −26 tumor-bearing mice. Control, tumor-free mice. (a) and (b) denote significant difference from control group and vehicle group, respectively (Kruskal-Wallis test, with Dunn's multiple-comparison test using Bonferroni adjustment, p<0.05). FIG. 6E shown the effects of MDP-H on muscle fiber size in C-26 tumor-bearing mice. The cross-sectional areas of muscle fibers in gastrocnemius muscles represented as a frequency histogram. Five sections from the gastrocnemius obtained from each of five mice per treatment group were analyzed as described in the Methods section. Using multiple comparisons for the log-rank test, comparison between muscles from tumorbearing/vehicle and tumor-bearing/MDP mice showed statistical significance (P<0.001). Data are presented as means. FIG. 6F shown the effects of MDP on grip strength.

As shown in FIG. 6A, MDP-H showed protecting mice from C-26 tumor-induced body weight loss on tumor burden at day 17.

As shown in FIG. 6B, MDP-H showed protecting mice from C-26 tumor-induced body weight loss (without tumor) on tumor burden at day 17.

As shown in FIG. 6C, MDP-H showed no significant suppressive effect on tumor burden at day 17.

As shown in FIG. 6D, MDP-H showed its ability to diminish cachexia-associated decreases in skeletal muscle weights.

As shown in FIG. 6E, MDP-H was able to rescue the fiber size distribution from shifting to smaller cross-sectional areas in cachectic muscles through immunostaining with anti-dystrophin of GC myofibers followed by quantification of myofiber diameter (P<0.0001).

As shown in FIG. 6F, MDP-H was able to restore the forelimb grip strength at day 17 (P<0.001).

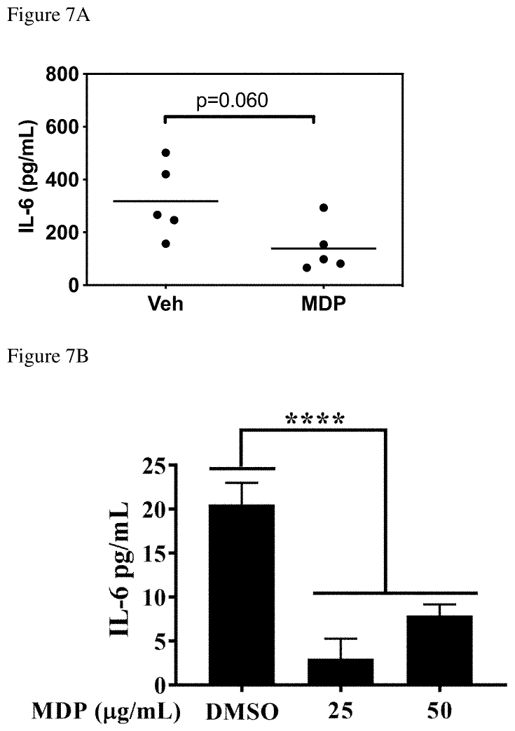

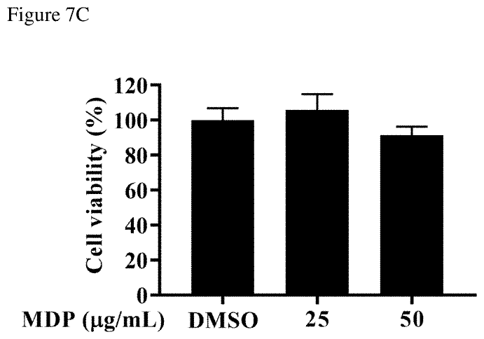

FIG. 7 shown the effect of MDP on serum IL-6 in Veh-versus MDP-H-treated C26-tumor-bearing mice. Wilcoxon rank sum test was used for statistical analysis (n=5). FIG. 7B shown the effects of MDP at 25 and 50 μg/mL on IL-6 production in C26 cell culture medium and FIG. 7C shown the viability of C26 cells. Bar, means±S.D. (for data obtained from three independent experiments for (B) and (C), where ****, P<0.0001.

As shown in FIG. 7A, the mean serum IL-6 (a major driver in the C-26 tumor model of cachexia) levels in MDP-H-treated C-26 tumor-bearing mice was lower but no statistically significant different from that of vehicle control (P=0.06).

As shown in FIG. 7B, the diminished serum IL-6 level was associated with the unique ability of MDP at 25 and 50 μg/mL to suppress IL-6 secretion into culture medium by C26 cells (P<0.001).

As shown in FIG. 7C, MDP at 25 and 50 μg/mL was non-cytotoxic to C26 cells.

### Example 4: Whole Transcriptome Shotgun Sequencing (RNA-Seq) Analysis

Whole transcriptome shotgun sequencing (RNA-seq) analysis was conducted by a commercial vendor (Welgene Biotech; Taiwan). Subsequently, these RNA-seq data were subjected to principal component analysis (PCA) to interrogate transcriptome variations among these groups. This clustering of expression profiles suggests that MDP was able to shift the gene expression profile of cachectic skeletal muscles (T/Veh) to a state similar to that of non-cachectic muscles (TF/Veh). Furthermore, pair comparisons of RNA-seq data was analyzed the differences in gene expression profiles among individual groups. Kyoto Encyclopedia of Genes and Genomes (KEGG) pathway and Gene Ontology (GO) Knowledgebase described the related biological signaling pathways.

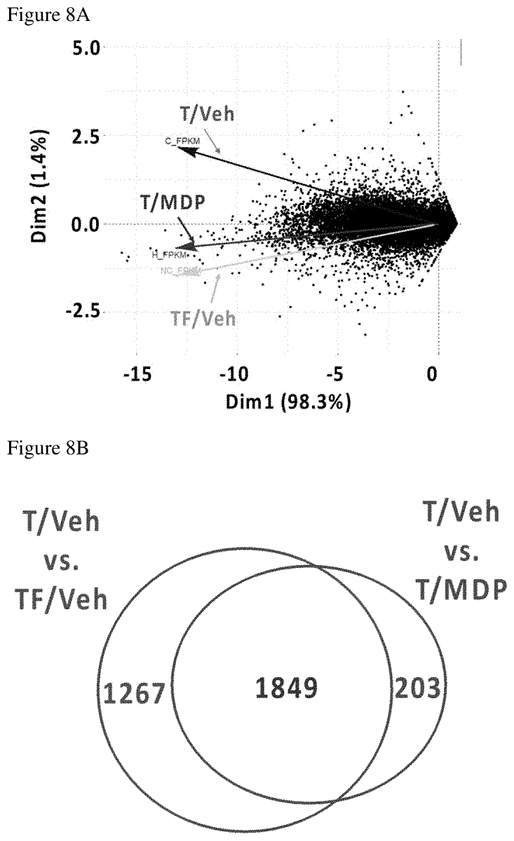

As shown in FIG. 8A, two-dimensional projection of RNA-seq data from the three study groups. The principal component analysis axes (Dim1, x-axis; Dim2, y-axis) emphasize the overall variation in RNA-seq data. As shown in FIG. 8B, each black dot represents a transformed gene expression value. Venn diagram of differentially expressed genes in each of the two pairwise comparisons of T/Veh versus TF/Veh and T/Veh versus T/MDP.

As shown in FIG. 8A, the principal component analysis (PCA) plot shows that the two-dimensional projection of the variation in the T/MDP group was much closer to that of the TF/Veh group than to that of the T/Veh group.

As shown in FIG. 8B, Venn diagram analysis reveals a total of 1849 differentially expressed genes shared by the two pairwise comparisons (center portion) that showed changed expression in the same direction.

The above bioinformatic analyses demonstrated the ability of MDP to reprogram the expression of genes associated with muscle homeostasis in cachectic skeletal muscles, which are reflected by the top twenty most up-versus down-regulated genes of the following Table 1 and Table 2.

TABLE 1
Top 20 most upregulated genes in skeletal muscles of MDP-
versus vehicle-treated C-26 tumor-bearing mice.

| Log2 | | | | |
|---|---|---|---|---|
| ratio | | | | |
| (MDP/ | NCBI | Gene | Gene | |
| Veh) | gene ID | name | description | Gene functions |
| 17.3 | 100504362 | Ccl21a | chemokine | Ccl21 acts as a chemoattractant of |
| | | | (C-C motif) | many types of immune cells via |
| | | | ligand 21A | the cell surface receptor CCR7. |
| | | | (serine) | CCR7 plays a role in regulating |
| | | | | energy metabolism by |
| | | | | suppressing brown adipose |
| | | | | tissues, which is involved in the |
| | | | | development of cancer cachexia |
| 15.7 | 20293 | Ccl12 | chemokine | Ccl12/MCP-5 specifically |
| | | | (C-C motif) | attracts eosinophils, monocytes |
| | | | ligand 12; | and lymphocytes by signaling |
| | | | aka, | through the receptor CCR2, |
| | | | monocyte | which plays a critical role in |
| | | | chemotactic | muscle regeneration following |
| | | | protein 5 | injury. |
| | | | (a.k.a., | |
| | | | MCP-5) | |
| 15.7 | 16846 | Lep | leptin | Deficiency in leptin has been |
| | | | | associated with reduced skeletal |
| | | | | muscle mass in genetically |
| | | | | engineered mice, and treatment |
| | | | | with exogenous leptin could |
| | | | | reverse the muscle loss by |
| | | | | inhibiting myofibrillar protein |
| | | | | degradation as well as enhancing |
| | | | | muscle cell proliferation |
| 6.63 | 16545 | Kera | Keratocan | One of the small leucine-rich |
| | | | | repeat proteoglycans (SLRPs) |
| | | | | identified as FoxO-regulated |
| | | | | transcripts downregulated in |
| | | | | cachectic muscle; located in the |
| | | | | ECM where they regulate the |
| | | | | structure and integrity of the |
| | | | | ECM. |
| 6.61 | 545798 | Tmem233 | Transmembrane | Highly and specifically expressed |
| | | | protein 233 | in skeletal muscles, of which the |
| | | | | functions remain unclear. |
| 6.05 | 12643 | Chad | Chondroadheri | One of the small leucine-rich |
| | | | | repeat proteoglycans (SLRPs) |
| | | | | identified as FoxO-regulated |
| | | | | transcripts downregulated in |
| | | | | cachectic muscle; located in the |
| | | | | ECM where they regulate the |
| | | | | structure and integrity of the |
| | | | | ECM. Chad is one of the most |
| | | | | downregulated genes in the |
| | | | | skeletal muscles of C26 tumor- |
| | | | | bearing mice |
| 5.50 | 16716 | Ky | Kyphoscoliosis | This muscle-specific protein |
| | | | peptidase | plays a vital role in muscle |
| | | | (Ky) | growth; the absence of Ky protein |
| | | | | leads to muscular dystrophy. |
| 5.46 | 68709 | Clip | Cartilage | The two isoforms of Cilp (Cilp-1 |
| | | | intermediate | and Cilp-2), are components of |
| | | | layer | extracellular matrix of cartilage, |
| | | | protein | which play a role in cartilage |
| | | | (Clip) | scaffolding. Downregulation of |
| | | | | Cilp is involved in joint |
| | | | | pathologies such as osteoarthritis. |
| | | | | Cilp is one of the most |
| | | | | downregulated genes in the |
| | | | | skeletal muscles of C26 tumor- |
| | | | | bearing mice. |
| 5.16 | 403183 | Mettl21e | Methyltransferase | This skeletal muscle-specific |
| | | | like 21E | lysine methyltransferase acts an |
| | | | | important modulator of |
| | | | | autophagy-associated protein |
| | | | | degradation in skeletal muscles, |
| | | | | and ablation of Mettl21c in mice |
| | | | | results in muscle weakness and |
| | | | | disturbance of the protein |
| | | | | degradation machinery. |
| 5.15 | 238564 | Mylk4 | Myosin | One of the members of the |
| | | | light chain | Myosin light chain kinase |
| | | | kinase | (MLCK) family which act as |
| | | | family, | regulatory proteins in smooth |
| | | | member 4 | muscle contraction. MYLK4 |
| | | | | expression was reported to |
| | | | | increase in skeletal muscles after |
| | | | | high-intensity intermittent |
| | | | | exercise training. |
| 5.12 | 66733 | Kcng4 | Potassium | Kcng4 is one of the most |
| | | | voltage- | downregulated genes in the |
| | | | gated | skeletal muscles of C26 tumor- |
| | | | channel, | bearing mice. Voltagegated |
| | | | subfamily | potassium (Kv) channels regulate |
| | | | G, member | diverse physiological functions, |
| | | | 4 | including neurotransmitter |
| | | | | release, heart rate, insulin |
| | | | | secretion, neuronal excitability, |
| | | | | epithelial electrolyte transport, |
| | | | | smooth muscle contraction, and |
| | | | | cell volume. |
| 4.90 | 18802 | Plcd4 | Phospholipase C, | Plcd4 is one of the most |
| | | | delta 4 | downregulated genes in the |
| | | | | skeletal muscles of C26 tumor- |
| | | | | bearing mice. Most abundant in |
| | | | | skeletal muscles, and responsible |
| | | | | for the production of important |
| | | | | second messengers, including |
| | | | | inositol trisphosphate and |
| | | | | diacylglycerol. |
| 4.80 | | Vwa3b | von | Intracellular proteins with VWA |
| | | | Willebrand | domains are thought to function |
| | | | factor A | in transcription, DNA repair, |
| | | | domain | ribosomal and membrane |
| | | | containing | transport, and the proteasome. |
| | | | 3B | Mutations in this gene are |
| | | | | associated with Spinocerebellar |
| | | | | ataxia. |
| 4.51 | 103968 | Plin1 | Perilipin 1 | Perilipin 1 promotes |
| | | | | triacylglycerol storage under |
| | | | | basal conditions by reducing the |
| | | | | access of cytosolic lipases to |
| | | | | triacylglycerol substrates stored |
| | | | | in lipid droplets. Expression of |
| | | | | perilipins in human skeletal |
| | | | | muscle in vitro and in vivo in |
| | | | | relation to diet, exercise and |
| | | | | energy balance. |
| 4.49 | 12377 | Casq1 | Calsequestrin 1 | A Ca2+-binding protein that acts |
| | | | | as a Ca2+ buffer within the |
| | | | | sarcoplasmic reticulum (SR), |
| | | | | helping storing Ca2+ in the |
| | | | | cisterna of the SR after muscle |
| | | | | contractions. |
| 4.48 | 16420 | Itgb6 | Integrin | Itgb6 mRNA was found highly |
| | | | beta 6 | enriched in skeletal muscles. |
| | | | | Evidence suggests that Itgb6 |
| | | | | protein is upregulated post- |
| | | | | transcriptionally in response to |
| | | | | muscle injury, which might be |
| | | | | involved in the remodeling of |
| | | | | extracellular matrix via TGF |
| | | | | signaling. |
| 4.43 | 64103 | Tnmd | Tenomodulin | A marker of tendons and |
| | | | | ligaments that integrate |
| | | | | musculoskeletal components, and |
| | | | | its loss-of-function in mice leads |
| | | | | to a phenotype with distinct signs |
| | | | | of premature aging at the tissue |
| | | | | and stem/progenitor cell levels. |
| 4.37 | 17306 | Sypl2 | Synaptophy- | SYPL2 is thought to participate in |
| | | | sin-like 2 | the excitation-contraction |
| | | | | coupling process of skeletal |
| | | | | muscle as SYPL2-null mice |
| | | | | showed reduced muscle |
| | | | | contractile force and altered triad |
| | | | | junction structure and increased |
| | | | | susceptibility to fatigue of the |
| | | | | skeletal muscle. |
| 4.32 | 74843 | Mss51 | MSS51 | Mss51 was predominantly |
| | | | mitochondrial | expressed in skeletal muscles and |
| | | | translationa1 | in those muscles dominated by |
| | | | activator | fast-Twitch fibers. In vitro, its |
| | | | | expression was upregulated upon |
| | | | | differentiation of C2C12 |
| | | | | myoblasts into myotubes. |
| 4.22 | 433294 | Mettl21c | Methyltransferase | Ablation of Mettl21c in mice |
| | | | like 21C | resulted in muscle weakness and |
| | | | | disturbance of the protein |
| | | | | degradation machinery. |

TABLE 2
Top 20 most downregulated genes in skeletal muscles of
MDP-versus vehicle-treated C-26 tumor-bearing mice.

| Log2 | | | | |
|---|---|---|---|---|
| ratio | | | | |
| (MDP/ | NCBI | Gene | Gene | |
| Veh) | gene ID | name | description | Gene functions |
| −7.43 | 237320 | Aldh8a1 | Aldehyde | ALDH8A1 is involved in |
| | | | dehydrogenase | retinaldehyde metabolism, |
| | | | 8 | specifically the 9-cis retinal, and |
| | | | family, | in oxidation of aliphatic |
| | | | member A1 | aldehydes and glutaraldehyde. |
| | | | | Although ALDHs are reported to |
| | | | | regulate skeletal muscle |
| | | | | homeostasis in healthy |
| | | | | individuals and patients with |
| | | | | Duchenne muscular dystrophy, |
| | | | | the exact role of ALDH8A1 in |
| | | | | cachectic muscles remains |
| | | | | unclear. |
| −5.85 | 16819 | Lcn2 | Lipocalin 2 | One of the most upregulated |
| | | | | genes in the skeletal muscles of |
| | | | | C26 tumorbearing mice. |
| | | | | Lipocalin was recently reported |
| | | | | to be a pathologic mediator of |
| | | | | cachexia through the |
| | | | | melanocortin 4 receptor in the |
| | | | | mediobasal hypothalamus. Its |
| | | | | expression is closely associated |
| | | | | with reduced food consumption |
| | | | | and lean mass loss, and lipocalin |
| | | | | 2 knockout mice are protected |
| | | | | from cachexia. |
| −5.07 | 17750 | Mt2 | Metallothionein | One of the most upregulated |
| | | | | genes in the skeletal muscles of |
| | | | | C26 tumorbearing mice. |
| | | | | Concomitant abrogation of |
| | | | | metallothioneins 1 and 2 resulted |
| | | | | in activation of the Akt pathway |
| | | | | and increases in myotube size, |
| | | | | and ultimately in muscle strength |
| | | | | mass and strength. |
| −5.04 | 13419 | Dnase1 | DNase I | The functional role of DNase I in |
| | | | | cachectic muscles remains |
| | | | | uncharacterized |
| −4.83 | 17748 | Mt1 | Metallothionein | Concomitant abrogation of |
| | | | 1 | metallothioneins 1 and 2 resulted |
| | | | | in activation of the Akt pathway |
| | | | | and increases in myotube size, |
| | | | | and ultimately in muscle strength |
| | | | | mass and strength. |
| −4.51 | 213053 | Slc39a14 | Solute | ZIP14 regulates zinc homeostasis |
| | | | carrier | in skeletal muscle, and represents |
| | | | family 39 | a critical mediator of cancer- |
| | | | (zinc | induced cachexia by facilitating |
| | | | transporter), | zinc accumulation, leading to |
| | | | member | muscle atrophy and blocked |
| | | | 14 (a.k.a., | muscle regeneration. |
| | | | ZIP14) | |
| −4.49 | 236690 | Nyx | Nyctalopin | Nyctalopin is one of the SLRP |
| | | | | members. Although it was |
| | | | | reported to be essential for |
| | | | | synaptic transmission in the cone |
| | | | | dominated zebrafish retina, its |
| | | | | role in cachectic muscles remains |
| | | | | uncharacterized. |
| −4.37 | 74127 | Krt80 | Keratin 80 | KRT80 is a filament protein that |
| | | | | make up one of the major |
| | | | | structural fibers of epithelial |
| | | | | cells. However, its role in |
| | | | | cachectic muscles remains |
| | | | | uncharacterized. |
| −4.29 | 20717 | Serpina3m | Serine (or | One of the most upregulated |
| | | | cysteine) | genes in the skeletal muscles of |
| | | | peptidase | C26 tumor bearing mice. |
| | | | inhibitor, | Serpina3m is likely involved in |
| | | | clade A, | neuromuscular junction |
| | | | member | maintenance and/or stability, as |
| | | | 3M | its expression is induced and |
| | | | | localized at the motor endplate |
| | | | | following denervation, and this |
| | | | | effect is augmented in a rodent |
| | | | | model of enhanced reinnervation. |
| −4.22 | 16529 | Kcnk5 | Potassium | Inhibition of TASK2 during |
| | | | channel, | differentiation revealed a |
| | | | subfamily | morphological impairment of |
| | | | K, member | myoblast fusion accompanied by |
| | | | 5 (a.k.a., | a downregulation of maturation |
| | | | TASK2) | markers in C2C12 cells. |
| −4.11 | 55985 | Cxcl13 | Chemokine | A significant upregulation of |
| | | | (C-X-C | CXCL13 was found in the CNS |
| | | | motif) | of the amyotrophic lateral |
| | | | ligand 13 | sclerosis mice, indicating a direct |
| | | | | correlation between the activation |
| | | | | of the chemokine and a faster |
| | | | | disease progression. |
| −4.00 | 216725 | Adamts2 | A | ADAM-TS2 is also known as |
| | | | disintegrinlike | procollagen I N-proteinase (PC I- |
| | | | and | NP). ADAMTS2 is responsible |
| | | | metallopeptidase | for processing several types of |
| | | | (reprolysin | procollagen proteins. |
| | | | type) with | Procollagens are the precursors of |
| | | | thrombospondin | collagens, the proteins that add |
| | | | type 1 | strength and support to many |
| | | | motif, 2 | body tissues. |
| | | | (ADAM-TS2) | |
| −3.93 | 11717 | Ampd3 | Adenosine | AMPD3 facilitates adenine |
| | | | monophosphate | nucleotide degradation in skeletal |
| | | | deaminase | muscles, which was found to |
| | | | 3 (AMPD3) | accelerate protein degradation in |
| | | | | C2C12 myotubesand to |
| | | | | contribute to the pathophysiology |
| | | | | of skeletal muscle atrophy. |
| −3.87 | 14085 | Fah | Fumarylacetoacetate | This gene encodes the last |
| | | | hydrolase | enzyme in the tyrosine |
| | | | | catabolism pathway, of which the |
| | | | | role in cachectic muscles remain |
| | | | | uncharacterized. |
| −3.81 | 13447 | Doc2b | Double C2, | One of the most upregulated |
| | | | beta | genes in the skeletal muscles of |
| | | | | C26 tumor bearing mice. Doc2b |
| | | | | is a key positive regulator of |
| | | | | Muncl8c-syntaxin 4-mediated |
| | | | | insulin secretion as well as of |
| | | | | insulin responsiveness in skeletal |
| | | | | muscle, and thus a key effector |
| | | | | for glucose homeostasis in vivo. |
| −3.69 | 93732 | Acox2 | Acyl- | ACOX2 encodes branched-chain |
| | | | Coenzyme | acylCoA oxidase, a peroxisomal |
| | | | A oxidase | enzyme believed to be involved |
| | | | 2, branched | in the metabolism of branched- |
| | | | chain | chain fatty acids and bile acid |
| | | | | intermediates |
| −3.66 | 432516 | Myo1a | Myosin IA | Myosins are a superfamily of |
| | | | | motor proteins best known for |
| | | | | their roles in muscle contraction |
| | | | | and in a wide range of other |
| | | | | motility processes in eukaryotes. |
| −3.65 | 233011 | Itpkc | Inositol | ITPKC catalyzes the |
| | | | 1,4,5- | phosphorylation of inositol 1,4,5- |
| | | | trisphosphate | trisphosphate to 1,3,4,5- |
| | | | 3-kinase | tetrakisphosphate, and has been |
| | | | C (ITPKC) | proposed as a prognostic and |
| | | | | predictive biomarker of |
| | | | | neoadjuvant chemotherapy for |
| | | | | triple negative breast cancer. |
| −3.52 | 68738 | Acss1 | Acyl-CoA | ACSS1 is a mitochondrial matrix |
| | | | synthetase | enzyme that is strongly expressed |
| | | | short-chain | in heart, skeletal muscle and |
| | | | family | brown adipose tissue. |
| | | | member 1 | |
| | | | (ACSS1) | |
| −3.41 | 16009 | Igfbp3 | Insulin-like | IGFBP-3 potently leads to |
| | | | growth | impaired myogenesis and |
| | | | factor | enhanced muscle protein |
| | | | binding | degradation, the major features of |
| | | | protein 3 | muscle wasting, via IGF |
| | | | (IGFBP-3) | signaling inhibition. |

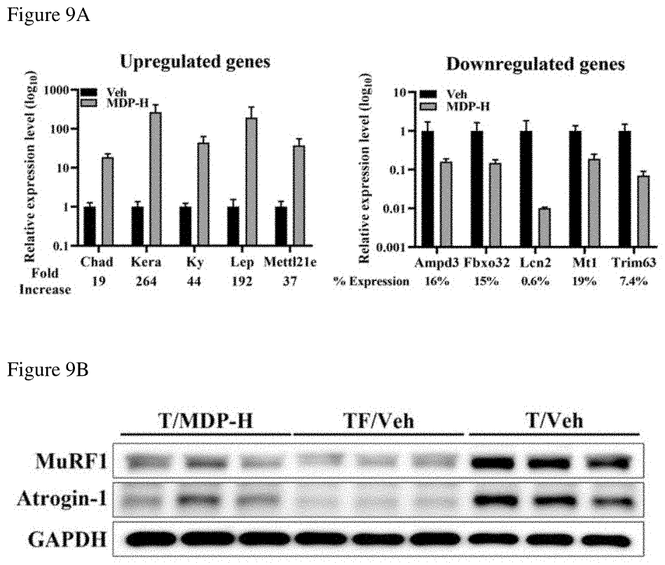

As shown in FIG. 9, the effects of MDP on skeletal muscle-related genes and protein expressions. As shown in FIG. 9A, 5 upregulated (left) and 5 downregulated (right) genes in skeletal muscles from vehicle-treated versus MDP-H-treated mice (n=3 for each group) were shown by qPCR analysis. The fold increase and % expression on upregulated and downregulated genes were shown as relative expression levels of selected skeletal muscle-related genes of MDP-H-treated mice to the vehicle counterparts, respectively. Bar, means+S.D (n=3). As shown in FIG. 9B, western blot analysis of the protein expression levels of MuRF1 and Atrogin-1 in skeletal muscles from vehicle- and MDP-H-treated C26 tumor-bearing mice (T/Veh and T/MDP-H, respectively) vis-à-vis vehicle-treated tumor-free mice (TF/Veh) were shown, respectively. The forward primer and reverse primer of the quantitative RT-PCR was shown in the following Table 3.

TABLE 3
Primer sequences used for quantitative
RT-PCR analysis

| Gene | Forward/ | |
|---|---|---|
| name | Reverse | Sequence (5’-3’) |
| 18s | Forward | AGAAACGGCTACCACATCCA |
| rRNA | | (SEQ ID NO: 1) |
| | Reverse | CCCTCCAATGGATCCTCGTT |
| | | (SEQ ID NO: 2) |
| Kera | Forward | CAGCCACAGGACTCAACGG |
| | | (SEQ ID NO: 3) |
| | Reverse | AGTAGGGAAACTGGGAGGACA |
| | | (SEQ ID NO: 4) |
| LEP | Forward | AAGGGGCTTGGGTTTTTCCA |
| | | (SEQ ID NO: 5) |
| | Reverse | CAGACAGAGCTGAGCACGAA |
| | | (SEQ ID NO: 6) |
| Ky | Forward | ACAGTCAATGGGAAAGCCACA |
| | | (SEQ ID NO: 7) |
| | Reverse | CTCCAGCTTCATCCCGTTCT |
| | | (SEQ ID NO: 8) |
| Chad | Forward | CAACTCGTTTCGGACCATGC |
| | | (SEQ ID NO: 9) |
| | Reverse | GATGTCGTTGTGGGACAGGT |
| | | (SEQ ID NO: 10) |
| Mettl21e | Forward | GCCATCGGCCCTTGTTCTAT |
| | | (SEQ ID NO: 11) |
| | Reverse | TAGCAATCACACGAGCACCA |
| | | (SEQ ID NO: 12) |
| Trim63 | Forward | AGGGACTAGCATAGGGCTCC |
| | | (SEQ ID NO: 13) |
| | Reverse | TGACAATCGCCAGTCACACA |
| | | (SEQ ID NO: 14) |
| Fbxo32 | Forward | CGGGGTTTGTTTTCAGCAGG |
| | | (SEQ ID NO: 15) |
| | Reverse | ACACAGACATTGCCTCCCAG |
| | | (SEQ ID NO: 16) |
| Lcn2 | Forward | TGAGTGTCATGTGTCTGGGC |
| | | (SEQ ID NO: 17) |
| | Reverse | GAACTGATCGCTCCGGAAGT |
| | | (SEQ ID NO: 18) |
| Mt1 | Forward | CTGTCCTCTAAGCGTCACCA |
| | | (SEQ ID NO: 19) |
| | Reverse | AGCAGCTCTTCTTGCAGGAG |
| | | (SEQ ID NO: 20) |
| Ampd3 | Forward | ACAACTGACCTGTCCTCCCT |
| | | (SEQ ID NO: 21) |
| | Reverse | CAAAGCTCAGCCCGTTAGGA |
| | | (SEQ ID NO: 22) |

As shown in FIG. 9A, the changes in the expression levels of upregulated and downregulated genes from qPCR analysis paralleled that of RNA-seq analysis.

As shown in FIG. 9B, MDP-H was effective in suppressing the protein expression of Atrogin-1 and MuRF1 in C26 tumor-bearing mice to the basal levels noted in the control group upon Western blotting analysis.

While the invention has been described and exemplified in sufficient details for those skilled in this art to make and use it, various alternatives, modifications, and improvements should be apparent without departing from the spirit and scope of this invention.

One skilled in the art readily appreciates that the present invention is well adapted to carry out the objects and obtain the ends and advantages mentioned, as well as those inherent therein. The processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

SEQUENCE LISTING

<ST26Sequencelisting dtdVersion = ″V1_3″ fileName = ″A TRADITIONAL CHINESE
MEDICINE-BASED AGENT FOR CACHEXIA PREVENTION AND
TREATMENT.xml″ softwareName = ″WIPO
Sequence″ softwareVersion = ″2.1.2″ productionDate = ″2022-10-13″>
<ApplicationIdentification>
<IPOfficeCode>US</IPOfficeCode>
<ApplicationNumberText/>
<FilingDate/>
</ApplicationIdentification>
<ApplicantFileReference>3991-CMU-US</ApplicantFileReference>
<EarliestPriorityApplicationIdentification>
<IPOfficeCode>US</IPOfficeCode>
<ApplicationNumberText>63/255,963</ApplicationNumberText>
<FilingDate>2021-10-15</FilingDate>
</EarliestPriorityApplicationldentification>
<ApplicantName languageCode = ″en″>CHINA MEDICAL UNIVERSITY</ApplicantName>
<InventorName languageCode = ″en″>Hsiang-Wen Lin</InventorName>
<InventorNameLatin>Hsiang-Wen Lin</InventorNameLatin>
<InventionTitle languageCode = ″en″>A TRADITIONAL CHINESE MEDICINE-BASED
AGENT FOR CACHEXIA PREVENTION AND TREATMENK/InventionTitle>
<SequenceTotalQuantity>22</SequenceTotalQuantity>
<SequenceData sequenceIDNumber = ″1″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..20</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q3″>
<INSDQualifier_name>note</INSDQualifier name>
<INSDQualifier_value>forward primer</INSDQualifier_value>
</INSDQualifier>
<INSDQualifier id = ″q1″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>agaaacggctaccacatcca</INSDSeq_sequence>
</INSDSeq>
</SequenceData>
<SequenceData sequenceIDNumber = ″2″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..20</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q4″>
<INSDQualifier_name>note</INSDQualifier_name>
<INSDQualifier_value>reverse primer</INSDQualifier_value>
</INSDQualifier>
<INSDQualifier id = ″q2″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>ccctccaatggatcctcgtt</INSDSeq_sequence>
</INSDSeq>
</SequenceData>
<SequenceData sequenceIDNumber = ″3″>
<INSDSeq>
<INSDSeq_length>19</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..19</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q6″>
<INSDQualifier_name>note</INSDQualifier_name>
<INSDQualifier_value>forward primer</INSDQualifier_value>
</INSDQualifier>
<INSDQualifier id = ″q5″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>cagccacaggactcaacgg</INSDSeq_sequence>
</INSDSeq>
</SequenceData>
<SequenceData sequenceIDNumber = ″4″>
<INSDSeq>
<INSDSeq_length>21</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..21</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q8″>
<INSDQualifier_name>note</INSDQualifier_name>
<INSDQualifier_value>reverse primer</INSDQualifier_value>
</INSDQualifier>
<INSDQualifier id = ″q7″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>agtagggaaactgggaggaca</INSDSeq_sequence>
</INSDSeq>
</SequenceData>
<SequenceData sequenceIDNumber = ″5″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..20</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q10″>
<INSDQualifier_name>note</INSDQualifier_name>
<INSDQualifier_value>forward primer</INSDQualifier_value>
</INSDQualifier>
<INSDQualifier id = ″q9″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>aaggggcttgggtttttcca</INSDSeq_sequence>
</INSDSeq>
</SequenceData>
<SequenceData sequenceIDNumber = ″6″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..20</INSDFeature_location>
<INSDFeature_quals>
<INSDQualifier>
<INSDQualifier_name>mol type</INSDQualifier_name>
<INSDQualifier_value>other DNA</INSDQualifier_value>
</INSDQualifier>
<INSDQualifier id = ″q15″>
<INSDQualifier_name>note</INSDQualifier_name>
<INSDQualifier_value>reverse primer</INSDQualifier_value>
</INSDQualifier>
<INSDQualifier id = ″q14″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>cagacagagctgagcacgaa</INSDSeq_sequence>
</INSDSeq>
</SequenceData>
<SequenceData_sequenceIDNumber = ″7″>
<INSDSeq>
<INSDSeq_length>21</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..21</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q17″>
<INSDQualifier_name>note</INSDQualifier_name>
<INSDQualifier_value>forward primer</INSDQualifier_value>
</INSDQualifier>
<INSDQualifier id = ″q16″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>acagtcaatgggaaagccaca</INSDSeq_sequence>
</INSDSeq>
</SequenceData>
<SequenceData_sequenceIDNumber = ″8″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..20</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q19″>
<INSDQualifier_name>note</INSDQualifier_name>
<INSDQualifier_value>reverse primer</INSDQualifier_value>
</INSDQualifier>
<INSDQualifier id = ″q18″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>ctccagcttcatcccgttct</INSDSeq_sequence>
</INSDSeq>
</SequenceData>
<SequenceData sequenceIDNumber = ″9″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..20</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q21″>
<INSDQualifier_name>note</INSDQualifier_name>
<INSDQualifier_value>forward primer</INSDQualifier_value>
</INSDQualifier>
<INSDQualifier id = ″q20″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>caactcgtttcggaccatgc</INSDSeq_sequence>
</INSDSeq>
</SequenceData>
<SequenceData sequenceIDNumber = ″10″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..20</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q23″>
<INSDQualifier_name>note</INSDQualifier_name>
<INSDQualifier_value>reverse primer</INSDQualifier_value>
</INSDQualifier>
<INSDQualifier id = ″q22″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>gatgtcgttgtgggacaggt</INSDSeq_sequence>
</INSDSeq>
</SequenceData>
<SequenceData sequenceIDNumber = ″11″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..20</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q25″>
<INSDQualifier_name>note</INSDQualifier_name>
<INSDQualifier_value>forward primer</INSDQualifier_value>
</INSDQualifier>
<INSDQualifier id = ″q24″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>gccatcggcccttgttctat</INSDSeq_sequence>
</INSDSeq>
</SequenceData>
<SequenceData sequenceIDNumber = ″12″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..20</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q27″>
<INSDQualifier_name>note</INSDQualifier_name>
<INSDQualifier_value>reverse primer</INSDQualifier_value>
</INSDQualifier>
<INSDQualifier id = ″q26″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>tagcaatcacacgagcacca</INSDSeq_sequence>
</INSDSeq>
</SequenceData>
<SequenceData sequenceIDNumber = ″13″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..20</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q29″>
<INSDQualifier_name>note</INSDQualifier_name>
<INSDQualifier_value>forward primer</INSDQualifier_value>
</INSDQualifier>
<INSDQualifier id = ″q28″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>agggactagcatagggctcc</INSDSeq_sequence>
</INSDSeq>
</SequenceData>
<SequenceData sequenceIDNumber = ″14″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..20</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q31″>
<INSDQualifier_name>note</INSDQualifier_name>
<INSDQualifier_value>reverse primer</INSDQualifier_value>
</INSDQualifier>
<INSDQualifier id = ″q30″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>tgacaatcgccagtcacaca</INSDSeq_sequence>
</INSDSeq>
</SequenceData>
<SequenceData sequenceIDNumber = ″15″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..20</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q33″>
<INSDQualifier_name>note</INSDQualifier_name>
<INSDQualifier_value>forward primer</INSDQualifier_value>
</INSDQualifier>
<INSDQualifier id = ″q32″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>cggggtttgttttcagcagg</INSDSeq_sequence>
</lNSDSeq>
</SequenceData>
<SequenceData sequenceIDNumber = ″16″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..20</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q35″>
<INSDQualifier_name>note</INSDQualifier_name>
<INSDQualifier_value>reverse primer</INSDQualifier_value>
</INSDQualifier>
<INSDQualifier id = ″q34″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>acacagacattgcctcccag</INSDSeq_sequence>
</INSDSeq>
</SequenceData>
<SequenceData sequenceIDNumber = ″17″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..20</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q37″>
<INSDQualifier_name>note</INSDQualifier_name>
<INSDQualifier_value>forward primer</INSDQualifier_value>
</INSDQualifier>
<INSDQualifier id = ″q36″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>tgagtgtcatgtgtctgggc</INSDSeq_sequence>
</INSDSeq>
</SequenceData>
<SequenceData sequenceIDNumber = ″18″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..20</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q39″>
<INSDQualifier_name>note</INSDQualifier_name>
<INSDQualifier_value>reverse primer</INSDQualifier_value>
</INSDQualifier>
<INSDQualifier id = ″q38″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>gaactgatcgctccggaagt</INSDSeq_sequence>
</INSDSeq>
</SequenceData>
<SequenceData sequenceIDNumber = ″19″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..20</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q41″>
<INSDQualifier_name>note</INSDQualifier_name>
<INSDQualifier_value>forward primer</INSDQualifier_value>
</INSDQualifier>
<INSDQualifier id = ″q40″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>ctgtcctctaagcgtcacca</INSDSeq_sequence>
</INSDSeq>
</SequenceData>
<SequenceData sequenceIDNumber = ″20″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..20</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q43″>
<INSDQualifier_name>note</INSDQualifier_name>
<INSDQualifier_value>reverse primer</INSDQualifier_value>
</INSDQualifier>
<INSDQualifier id = ″q42″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>agcagctcttcttgcaggag</INSDSeq_sequence>
</INSDSeq>
</SequenceData>
<SequenceData sequenceIDNumber = ″21″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..20</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q45″>
<INSDQualifier_name>note</INSDQualifier_name>
<INSDQualifier_value>forward primer</INSDQualifier_value>
</INSDQualifier>
<INSDQualifier id = ″q44″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>acaactgacctgtcctccct</INSDSeq_sequence>
</INSDSeq>
</SequenceData>
<SequenceData sequenceIDNumber = ″22″>
<INSDSeq>
<INSDSeq_length>20</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>l..20</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q47″>
<INSDQualifier_name>note</INSDQualifier_name>
<INSDQualifier_value>reverse primer</INSDQualifier_value>
</INSDQualifier>
<INSDQualifier id = ″q46″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>caaagctcagcccgttagga</INSDSeq_sequence>
</INSDSeq>
</SequenceData>
</ST26SequenceListing>

What is claimed is:

1. A method of treating or preventing cancer-induced cachexia, comprising the administration of effective amounts of a composition to a subject with cancer-induced cachexia, wherein the composition comprises a Moutan radicis cort or a Moutan radicis cort extract.

2. The method of claim 1, wherein the Moutan radicis cort extract is prepared by soaking the Moutan radicis cort in 50% to 100% methanol or 50% to 100% ethanol.

3. The method of claim 2, wherein the Moutan radicis cort extract is prepared by soaking the Moutan radicis cort in 70% methanol or 70% ethanol.

4. A method of treating or preventing cancer-induced skeletal muscle weight losses, comprising the administration of effective amounts of a composition to a subject with cancer-induced muscle atrophy or muscle weight loss, wherein the composition comprises a Moutan radicis cort or a Moutan radicis cort extract.

5. The method of claim 4, wherein the Moutan radicis cort extract is prepared by soaking the Moutan radicis cort in 50% to 100% methanol or 50% to 100% ethanol.

6. The method of claim 5, wherein the Moutan radicis cort extract is prepared by soaking the Moutan radicis cort in 70% methanol or 70% ethanol.

7. The method of claim 4, wherein the composition upregulates the muscle homeostasis-associated gene, comprising Cc121a, Cc112, Lep, Kera, Tmem233, Chad, Ky, Clip, Mett121e, Mylk4, Kcng4, Plcd4, Vwa3b, Plin1, Casq1, Itgb6, Tnmd, Syp12, Mss51, Mett121c. 27 or combination thereof to reduce cancer-induced muscle atrophy.

8. The method of claim 4, wherein the composition downregulates the muscle homeostasis-associated gene, comprising Aldh8a1, Lcn2, Mt2, Dnase1, Mt1, Slc39a14, Nyx, Krt80, Serpina3m, Kcnk5, Cxcl13, Adamts2, Ampd3, Fah, Doc2b, Acox2, Myo1a, Itpkc, Acss1, Igfbp3. or combination thereof to reduce cancer-induced muscle atrophy.


SEQUENCE LISTING (22 sequences)

SEQ ID NO: 1 - DNA, 20 bp, synthetic construct
  source 1..20: mol_type = other DNA; note = forward primer
agaaacggctaccacatcca

SEQ ID NO: 2 - DNA, 20 bp, synthetic construct
  source 1..20: mol_type = other DNA; note = reverse primer
ccctccaatggatcctcgtt

SEQ ID NO: 3 - DNA, 19 bp, synthetic construct
  source 1..19: mol_type = other DNA; note = forward primer
cagccacaggactcaacgg

SEQ ID NO: 4 - DNA, 21 bp, synthetic construct
  source 1..21: mol_type = other DNA; note = reverse primer
agtagggaaactgggaggaca

SEQ ID NO: 5 - DNA, 20 bp, synthetic construct
  source 1..20: mol_type = other DNA; note = forward primer
aaggggcttgggtttttcca

SEQ ID NO: 6 - DNA, 20 bp, synthetic construct
  source 1..20: mol_type = other DNA; note = reverse primer
cagacagagctgagcacgaa

SEQ ID NO: 7 - DNA, 21 bp, synthetic construct
  source 1..21: mol_type = other DNA; note = forward primer
acagtcaatgggaaagccaca

SEQ ID NO: 8 - DNA, 20 bp, synthetic construct
  source 1..20: mol_type = other DNA; note = reverse primer
ctccagcttcatcccgttct

SEQ ID NO: 9 - DNA, 20 bp, synthetic construct
  source 1..20: mol_type = other DNA; note = forward primer
caactcgtttcggaccatgc

SEQ ID NO: 10 - DNA, 20 bp, synthetic construct
  source 1..20: mol_type = other DNA; note = reverse primer
gatgtcgttgtgggacaggt

SEQ ID NO: 11 - DNA, 20 bp, synthetic construct
  source 1..20: mol_type = other DNA; note = forward primer
gccatcggcccttgttctat

SEQ ID NO: 12 - DNA, 20 bp, synthetic construct
  source 1..20: mol_type = other DNA; note = reverse primer
tagcaatcacacgagcacca

SEQ ID NO: 13 - DNA, 20 bp, synthetic construct
  source 1..20: mol_type = other DNA; note = forward primer
agggactagcatagggctcc

SEQ ID NO: 14 - DNA, 20 bp, synthetic construct
  source 1..20: mol_type = other DNA; note = reverse primer
tgacaatcgccagtcacaca

SEQ ID NO: 15 - DNA, 20 bp, synthetic construct
  source 1..20: mol_type = other DNA; note = forward primer
cggggtttgttttcagcagg

SEQ ID NO: 16 - DNA, 20 bp, synthetic construct
  source 1..20: mol_type = other DNA; note = reverse primer
acacagacattgcctcccag

SEQ ID NO: 17 - DNA, 20 bp, synthetic construct
  source 1..20: mol_type = other DNA; note = forward primer
tgagtgtcatgtgtctgggc

SEQ ID NO: 18 - DNA, 20 bp, synthetic construct
  source 1..20: mol_type = other DNA; note = reverse primer
gaactgatcgctccggaagt

SEQ ID NO: 19 - DNA, 20 bp, synthetic construct
  source 1..20: mol_type = other DNA; note = forward primer
ctgtcctctaagcgtcacca

SEQ ID NO: 20 - DNA, 20 bp, synthetic construct
  source 1..20: mol_type = other DNA; note = reverse primer
agcagctcttcttgcaggag

SEQ ID NO: 21 - DNA, 20 bp, synthetic construct
  source 1..20: mol_type = other DNA; note = forward primer
acaactgacctgtcctccct

SEQ ID NO: 22 - DNA, 20 bp, synthetic construct
  source 1..20: mol_type = other DNA; note = reverse primer
caaagctcagcccgttagga